(19) United States
(12) Patent Application Publication
(10) Pub. No.: US 2022/0047154 A1
(43) Pub. Date: Feb. 17, 2022

(54) ENDOLUMINAL ROBOTIC SYSTEMS AND METHODS EMPLOYING CAPSULE IMAGING TECHNIQUES

(71) Applicant: Covidien LP, Mansfield, MA (US)

(72) Inventors: Scott J. Prior, Branford, CT (US); Arvind Rajagopalan Mohan, Dracut, MA (US); John W. Komp, Dillon, CO (US); William J. Peine, Ashland, MA (US); Scott E.M. Frushour, Boulder, CO (US); Anthony B. Ross, Boulder, CO (US)

(21) Appl. No.: 17/395,908

(22) Filed: Aug. 6, 2021

Related U.S. Application Data
(60) Provisional application No. 63/125,293, filed on Dec. 14, 2020.
(60) Provisional application No. 63/064,938, filed on Aug. 13, 2020.

(51) Int. Cl. A61B 1/04; A61B 1/267; A61B 1/00; G06T 7/00; A61B 34/10; A61B 34/30

(52) U.S. Cl. CPC A61B 1/041; A61B 1/2676; A61B 1/0005; G06T 7/0014; A61B 34/10; A61B 34/30; G06T 2207/30061; A61B 2034/107; A61B 2562/0223; A61B 2562/0219; G06T 2207/20084; G06T 2207/10028; A61B 2034/301

(57) ABSTRACT

A system and method for capturing in-vivo images of an endoluminal network and generating a pathway for directing an endoluminal robot to drive a catheter to a desired location.

### CROSS REFERENCE TO RELATED APPLICATIONS

This application claims the benefit of and priority to U.S. Provisional Patent Application Ser. No. 63/064,938, filed on Aug. 13, 2020, and to U.S. Provisional Patent Application Ser. No. 63/125,293, filed on Dec. 14, 2020, the entire contents of each of which are incorporated herein by reference.

### BACKGROUND

### Technical Field

The present disclosure relates to the field of endoluminal imaging and navigation.

### Description of Related Art

There are known several imaging techniques for the acquisition of images from within an endoluminal space. For example, an endoscope may be employed to capture images and video while navigating a lumen of the body. The endoscope is typically articulatable in at least one direction to enable close viewing of items of interest within the body. Such endoscopes may be inserted into naturally occurring openings of the body or may be inserted into a port or other access point mechanically formed in the patient. Regardless of the access, the endoscope provides real time images that can be analyzed to identify points of interest or those requiring diagnostic or therapeutic intervention.

More recently, ingestible capsule imaging devices have been developed. A capsule, unlike an endoscope is relatively small and can be swallowed by the patient. Once swallowed, the capsule captures a number of images and transmits the images to a recorder located outside the patient. Depending on the portion of the gastro-intestinal (GI) tract that is of interest, acquisition of the images can take 10 to 15 hours. The Pill cam generally relies on the natural motion muscular contractions and bodily processes to move through the GI tract.

While both of these technologies present incredible advancements for clinician in evaluating and treating patients, improvements are always desired.

### SUMMARY

One aspect of the disclosure is directed to an endoluminal navigation system including an imaging device configured for capturing images in a first direction and in a second direction substantially opposite the first in an endoluminal network, an image processing device configured to receive the captured images and compile the images into one or more alternative forms, the imaging processing device including a processor and memory, the memory storing thereon a software application that when executed by the processor. The endoluminal navigation system also reviews the captured and compiled images to identify areas of interest, constructs a three-dimensional (3D) model from the captured images, where the 3D model represents a fly-through view of the endoluminal network, and includes a display configured to receive compiled images or the 3D model and to present the compiled images or 3D model to provide views in both the first and the second directions, where the areas of interest are identified in the 3D model or images.

In aspects, the endoluminal system may include a position and orientation sensor associated with the imaging device.

In other aspects, the position and orientation of the sensor may be associated with images captured at a position and orientation and a timestamp for capture of the images.

In certain aspects, the position and orientation sensor may be a magnetic field detection sensor.

In other aspects, the position and orientation sensor may be an inertial monitoring unit.

In aspects, the position and orientation sensor may be a flex sensor.

In certain aspects, the endoluminal navigation system may include a speed sensor determining the speed at which the imaging deice is transiting the endoluminal network.

In aspects, the imagine device may be mounted on a bronchoscope.

In accordance with another aspect of the present disclosure, a method for driving an endoluminal robot includes capturing a plurality of in-vivo images of an endoluminal network, analyzing the plurality of captured images to identify one or more areas of interest within the endoluminal network, analyzing the plurality of captured images to identify a plurality of landmarks within the endoluminal network, generating a pathway plan through the endoluminal network to arrive at the one or more areas of interest, signaling an endoluminal robot to drive a catheter through the endoluminal network, following the pathway plan, to arrive at the area of interest, and performing a diagnostic or therapeutic procedure at the area of interest.

In aspects, the plurality of in-vivo images may be captured by one or more imagers in a capsule.

In certain aspects, the capsule may be navigated through the endoluminal network using a magnetic field generator.

In other aspects, the method may include stitching the plurality of captured images together to form a two-dimensional model of the endoluminal network.

In certain aspects, the method may include generating a three-dimensional (3D) model from the plurality of captured images.

In aspects, the method may include generating the pathway plan with reference to the 3D model.

In accordance with another aspect of the present disclosure, a method of endoluminal imaging includes inserting a bronchoscope having forward and backward imaging capability into an airway of a patient, navigating the bronchoscope into the airways and capturing a plurality of images in both a forward and a backward perspective, determining a position and orientation within the airways a which each of the plurality of images was captured, analyzing with an artificial intelligence the captured plurality of images to identify area of interest for performance of a diagnostic or therapeutic procedure, generating a three-dimensional (3D) model of the airways of the patient, generating a pathway plan through the airways of the patient, signaling an endoluminal robot to drive a catheter through the airways to the areas of interest, assessing the position of the catheter within the airways by comparison of real-time images with previously captured forward and backwards images, presenting one or more of the real-time images or the previously captured forward and backward images or the 3D model on a graphic user interface, and performing a diagnostic or therapeutic procedure at the area of interest.

In aspects, the captured forward and backward images may be captured by one or more imagers in a capsule.

In certain aspects, the capsule may be navigated through the endoluminal network using a magnetic field generator.

In other aspects, the method may include stitching the plurality of captured forward and backwards images together to form a two-dimensional model of the endoluminal network.

In certain aspects, the method may include generating a three-dimensional (3D) model from the plurality of captured forward and backward images.

In aspects, the method may include generating the pathway plan with reference to the 3D model.

### BRIEF DESCRIPTION OF THE DRAWINGS

FIG. 1 depicts the distal portion of an endoscope in accordance with the present disclosure;

FIG. 2 shows a schematic diagram of an in-vivo capsule imaging system according to an embodiment of the present disclosure;

FIG. 3 depicts an endo-luminal navigation system in accordance with the present disclosure;

FIG. 4 depicts the distal portion of an endoscope in accordance with the present disclosure;

FIG. 5 depicts a robotic endo-luminal navigation system in accordance with the present disclosure;

FIGS. 6A and 6B depicts motorized elements to drive a catheter in accordance with the present disclosure;

FIG. 7 depicts a user interface for reviewing images acquired by the endoscope of FIG. 1 or the capsule of FIG. 2;

FIG. 8 depicts a further user interface for reviewing images acquired by the endoscope of FIG. 1 or the capsule of FIG. 2; and

FIG. 9 depicts a flow chart of a method in accordance with the present disclosure.

### DETAILED DESCRIPTION

This disclosure relates to endo-luminal navigation and imaging. There exist systems and method for assessing the disease state of a patient using pre-procedural computed tomography (CT) or magnetic resonance imaging (MRI) image data sets. These pre-procedural image data sets are particularly beneficial for identifying tumors and lesions within the body of the patient.

While these pre-procedural extra-corporeal imaging techniques are very useful, they are of limited effect in assessing some of the common lung comorbidities. For example, many patients who suffer from lung cancer also suffer from diseases such as COPD and Emphysema. For these diseases in vivo images may be better for assessment of the condition of the patient, and importantly for monitoring the progression of the disease state as it is treated or progresses.

In view of these co-morbidities it can be challenging to identify locations for insertion of biopsy and therapeutic tools. Where the tissue is particularly damaged, the insertion of such tools can result in unintended damage to the luminal network of the patient. In-vivo imaging can be useful in identifying healthy or healthier tissue for insertion of such tools.

Additionally, extra-corporeal imaging has limits on the size the of the tumors and lesions. While in-vivo imaging will not likely reveal small tumors located outside of the airway walls, it will reveal small tumors and lesions that are located on the airway wall. The locations of these tumors and lesions can be marked such that they can be monitored and navigated to in the future.

Further, the images acquired by the in-vivo imaging system can be used to generate a three-dimensional (3D) model. Still further, artificial intelligence (AI) may be employed in the analysis of the in-vivo images to assist in identifying lesions and tumors.

Aspects of the present disclosure are directed to utilization of a bronchoscope or a capsule having capabilities to acquire images in both a forward and a rearward direction. These images are used in an initial diagnostic effort to determine where within the endoluminal network lesions or other pathologies may be located. Following an initial imaging a secondary catheter-based device may be inserted into the endoluminal network and navigated to the locations of the lesions or pathologies for acquisition of a biopsy, conducting therapy, or other purposes. These two navigations of the endoluminal network may be spaced temporally from one another or may be performed close in time to one another. The second catheter-based device may include imaging devices that can be used to confirm its location within the endoluminal network during navigation, acquire additional data, and to visualize the biopsy or therapy. These and other aspects of the present disclosure are described in greater detail below.

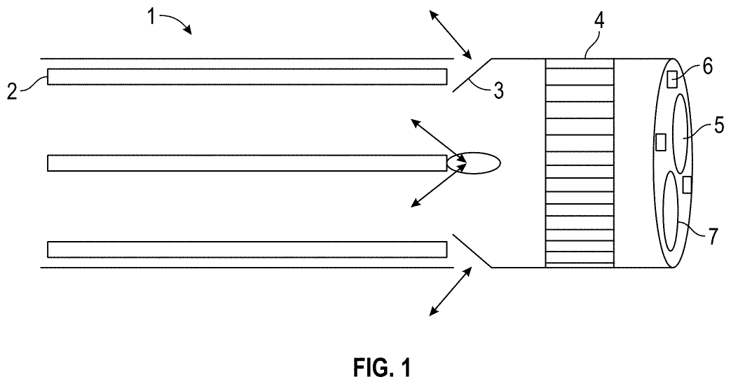

Reference is made to FIG. 1, which schematically illustrates an in-vivo imaging system according to an embodiment of the present disclosure. FIG. 1 depicts an endoscope 1 including a plurality of light pipes 2 and reflectors 3. The light pipes 2 and the reflectors 3 combine to project light travelling through the light pipes 2 to be reflected in a proximal direction. The reflectors 3 also collect light reflected from sidewalls of an endoluminal network and to be returned via a light pipe 2 to an image processing system as described in greater detail below. Certain of the light pipes 2 may be dedicated for projecting light into the endoluminal network and others dedicated to light capture for image creation. Alternatively, all of the light pipes 2 may be used for both light emission and light capture, for example by strobing light and capturing a reflection.

The endoscope 1 includes a position and orientation sensor 4 such as a magnetic field detection sensor, a flexible sensor to detect the shape of a distal portion of the endoscope 1, or an inertial measurement unit (IMU) or others. The sensor 4 provides an indication of where the distal portion of the endoscope 1 is at any time during a procedure.

A forward-looking imager 5 captures images of the endoluminal network in the forward direction as the endoscope 1 is advanced in the endo-luminal network. One or more light sources 6 provide for illumination of the endoluminal network in the forward direction to enable capture of the images. Again the light reflected from the sidewalls of the endoluminal network is captured by the imager 5 and may be converted immediately to an image (e.g., via complementary metal-oxide-semiconductor (CMOS) “camera on a chip”) and data representing the image is transmitted to an image processing system. Alternatively, the imager 5 is a lens connected via a light pipe (not shown) for conversion to an image via the image processor. In some embodiments a working channel 7 remains available for suction, lavage, or the passage of tools including biopsy and therapeutic tools, as described in greater detail below.

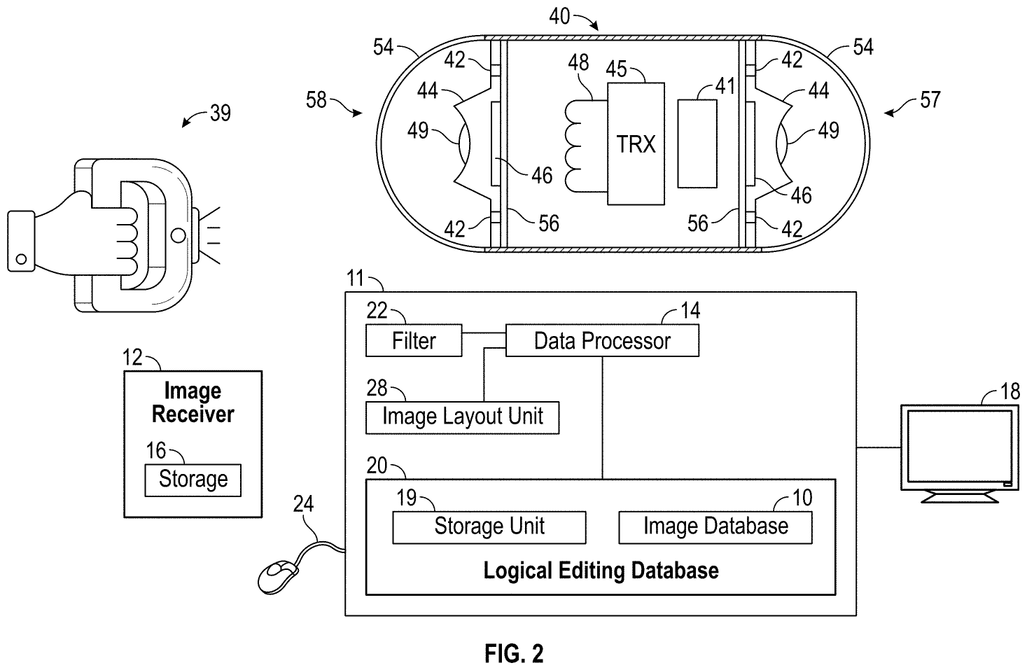

An alternative embodiment of the present disclosure is shown in FIG. 2 where the in-vivo imaging system is in the form of a capsule 40 may configured to communicate with an external receiving and display system to provide display of data, control, or other functions. Capsule 40 may include one or more imagers 46, for capturing images, one or more illumination sources 42, and a transmitter 41, for transmitting image data and possibly other information to a receiving device such as receiver 12. Transmitter 41 may include receiver capability, for example, to receive control information. In some embodiments, the receiver capability may be included in a separate component. An optical system, including, for example, lenses 49, lens holders 44 or mirrors, may aid in focusing reflected light onto the imagers 46. The lens holders 44, illumination units 42, and imagers 46 may be mounted on a substrate 56. An imaging head 57 and/or 58 may include the optical system, optical dome 54, imager 46, illumination units 42, and substrate 56. Power may be provided by an internal battery 45 or a wireless receiving system.

Both the catheter 1 and the capsule 40 are configured to communicate the acquired images outside of the patient's body to and image receiver 12, which may include an antenna or antenna array, an image receiver storage unit 16, a data processor 14, a data processor storage unit 19, and an display 18, for displaying, for example, the images recorded by the capsule 40.

According to embodiments of the present disclosure, data processor storage unit 19 may include an image database 10 and a logical editing database 20. Logical editing database 20 may include, for example, pre-defined criteria and rules for selecting images or portions thereof, stored in the image database 10, to be displayed to the viewer. In some embodiments, a list of the pre-defined criteria and rules may be displayed for selection by the viewer. In other embodiments, rules or criteria need not be selectable by a user. Examples of selection criteria may include, but are not limited to: average intensity of the image, average value of the R, B, or G pixels in the image, median value of the pixel intensity, criteria based on HSV color space, B/R, G/R, STD (standard deviation) values of the previous criteria, differences between images, etc. In some embodiments, a plurality of certain criteria may be associated to a rule or detector, for example, a polyp detector may use several criteria to determine whether a candidate polyp is present in the image. Similarly, a bleeding or redness detector may use different criteria to determine whether the image includes suspected bleeding or pathological tissue having an abnormal level of redness. In some embodiments, the user may decide which rules and/or detectors to activate.

According to a further aspect of the present disclosure, data processor 14, data processor storage unit 19 and display 18 are part of a personal computer or workstation 11 which includes standard components such as a processor, a memory, a disk drive, and input-output devices, although alternate configurations are possible, and the system and method of the present invention may be implemented on various suitable computing systems. An input device 24 may receive input from a user (e.g., via a pointing device, click-wheel or mouse, keys, touch screen, recorder/microphone, other input components) and send corresponding commands to trigger control of the computer components, e.g., data processor 14.

Data processor 14 may include one or more standard data processors, such as a microprocessor, multiprocessor, accelerator board, or any other serial or parallel high-performance data processor. Image monitor 18 may be a computer screen, a conventional video display, or any other device capable of providing image or other data.

As with the forward-facing imager 5 of FIG. 1, the imagers 46 may be formed of a suitable complementary metal-oxide-semiconductor (CMOS) camera, such as a “camera on a chip” type CMOS imager. In alternate embodiments, the imagers 46 may be another device, for example, a charge-coupled device (CCD). The illumination sources 42 may be, for example, one or more light emitting diodes, or another suitable light source.

During an in vivo imaging procedure, imagers 46 capture images and send data representing the images to transmitter 41, which transmits images to image receiver 12 using, for example, electromagnetic radio waves. Other signal transmission methods are possible and, alternatively, data may be downloaded from capsule 40 after the procedure. Further, with respect to the embodiment of FIG. 1, the imager 5 and the light pipe 2/reflector combinations may be directly connected to the image receiver 12 via a wired or wireless connection. Image receiver 12 may transfer the image data to image receiver storage unit 16. After a certain period of time of data collection, the image data stored in storage unit 16 may be sent to the data processor 14 or the data processor storage unit 19. For example, the image receiver storage unit 16 may be connected to the personal computer or workstation which includes the data processor 14 and data processor storage unit 19 via a standard data link, e.g., a USB interface of known construction. The image data may then be transferred from the image receiver storage unit 16 to the image database 10 within data processor storage unit 19. In other embodiments, the data may be transferred from the image receiver storage unit 16 to the image database 10 using a wireless communication protocol, such as Bluetooth, WLAN, or other wireless network protocols.

Data processor 14 may analyze and edit the data, for example, according to the logical editing database 20, and provide the analyzed and edited data to the display 18, where for example a health professional views the image data. Data processor 14 may operate software which, in conjunction with basic operating software such as an operating system and device drivers, controls the operation of data processor 14. According to one embodiment, the software controlling data processor 14 may include code written, for example, in the C++ language and possibly alternative or additional languages and may be implemented in a variety of known methods.

The image data collected and stored may be stored indefinitely, transferred to other locations, manipulated or analyzed. A health professional may use the images to diagnose pathological conditions of, for example, the GI tract, lungs or other endoluminal networks, and in addition, the system may provide information about the location of these pathologies. While using a system where the data processor storage unit 19 first collects data and then transfers data to the data processor 14, the image data may not be viewed in real time, other configurations allow for real time or quasi-real time viewing.

According to one embodiment, the imagers 46, (as well as imager 5 and the light pipe 2/reflector 3 combinations) may collect a series of still images as it traverses endoluminal network. The images may be later presented as, for example, a stream of images or a moving image of the traverse of the endoluminal network. One or more in-vivo imager systems may collect a large volume of data, as the capsule 40 may take some time to traverse the endoluminal network. The imagers 46 may record images at a rate of, for example, two to forty images per second (other rates, such as four frames per minute, may be used). The imagers 46 (as well as imager 5 and the light pipe 2/reflector 3 combinations) may have a fixed or variable frame capture and/or transmission rate. When the imagers 46 (as well as imager 5 and the light pipe 2/reflector 3 combinations) have a variable or adaptive frame rate (AFR), the imagers 46 (as well as imager 5 and the light pipe 2/reflector 3 combinations) may switch back and forth between frame rates, for example, based on parameters, such as the capsule 40 speed which may be detected by a speed sensor such as an inertial monitoring unit (IMU), capsule 40 estimated location, similarity between consecutive images, or other criteria. A total of thousands of images, for example, over 300,000 images, may be recorded. The image recordation rate, the frame capture rate, the total number of images captured, the total number of images selected for the edited moving image, and the view time of the edited moving image, may each be fixed or varied.

The image data recorded and transmitted by the capsule 40 or the endoscope 1 is digital color image data, although in alternate embodiments other image formats may be used. In an exemplary embodiment, each frame of image data includes 256 rows of 256 pixels each, each pixel including bytes for color and brightness, according to known methods. For example, in each pixel, color may be represented by a mosaic of four sub-pixels, each sub-pixel corresponding to primaries such as red, green, or blue (where one primary is represented twice). The brightness of the overall pixel may be recorded by a one byte (i.e., 0-255) brightness value. According to one embodiment, images may be stored sequentially in data processor storage unit 19. The stored data may include one or more pixel properties, including color and brightness.

While, information gathering, storage and processing are performed by certain units, the system and method of the present invention may be practiced with alternate configurations. For example, the components gathering image information need not be contained in a capsule, but may be contained in any other vehicle suitable for traversing a lumen in a human body, such as an endoscope, stent, catheter, needle, etc.

Data processor storage unit 19 may store a series of images recorded by a capsule 40 or endoscope 1. The images the capsule 40 or endoscope 1 records as it moves through a patient's endoluminal network may be combined by the data processor 14 consecutively to form a moving image stream or movie. Further, the images may be combined by the data processor 14 to form a 3D model of the endoluminal network that can be presented on the display 18 and provide a fly through view of the endoluminal network.

In an application where the endoluminal network is the airways of the lungs, the capsule 40 may formed in part of a ferrous material such that it may be impacted by magnetic fields. In order to navigate the capsule 40 through the airways, a hand-held or robotic magnetic field generator 39 may be placed proximate the capsule 40. Interaction of the magnetic field generated by the magnetic field generator enables the capsule 40 to be traversed through the airways. The images may be displayed on the display 18 as they are being captured by the capsule 40. Whether a handheld, motor driven, or robotic device, the magnetic field generator 39 can be manipulated to enable decisions to be made at each bifurcation of an endoluminal network (e.g., the airways). In this manner all of the airways of the lungs may be navigating up to the diameter of the capsule 40 and images may be acquired to generate a pre-procedure image data set. Details of the analysis of the image data set as well as 3D model generation is described in greater detail below.

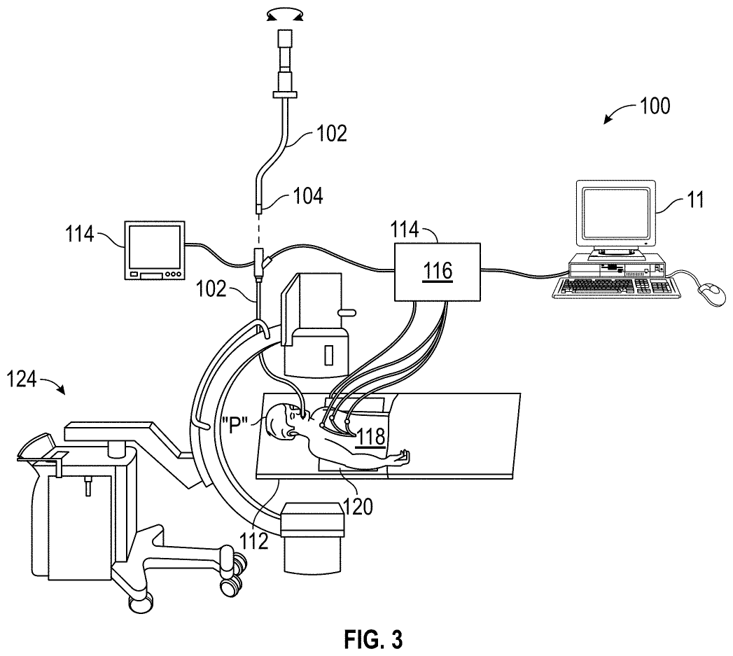

As shown in FIG. 3, bronchoscope 102 (e.g., endoscope 1) is configured for insertion into the mouth or nose of a patient “P”. A sensor 104 may be located on the distal portion of the bronchoscope 102. As described above, the position and orientation of sensor 104 relative to a reference coordinate system, and thus the distal portion of bronchoscope 102 can be derived.

System 100 generally includes an operating table 112 configured to support a patient P; tracking system 114 coupled to bronchoscope 102 (e.g., a video display, for displaying the video images received from the video imaging system of bronchoscope 102). The system 100 may optionally include a locating or tracking system 114 including a locating module 116. Where the locating or tracking system 114 is an electromagnetic system, system 100 may further include a plurality of reference sensors 118 and a transmitter mat 120 including a plurality of incorporated markers; and a computing device or workstation 11 including software and/or hardware used to facilitate identification of a target, pathway planning to the target, navigation of the bronchoscope 102 through the airways of the patient.

A fluoroscopic imaging device 124 capable of acquiring fluoroscopic or x-ray images or video of the patient P is also included in this particular aspect of system 100. The images, sequence of images, or video captured by fluoroscopic imaging device 124 may be stored within fluoroscopic imaging device 124 or transmitted to workstation 11 for storage, processing, and display. Additionally, fluoroscopic imaging device 124 may move relative to the patient P so that images may be acquired from different angles or perspectives relative to patient P to create a sequence of fluoroscopic images, such as a fluoroscopic video. The pose of fluoroscopic imaging device 124 relative to patient P and while capturing the images may be estimated via markers incorporated with the transmitter mat 120. The markers are positioned under patient P, between patient P and operating table 112 and between patient P and a radiation source or a sensing unit of fluoroscopic imaging device 124. The markers incorporated with the transmitter mat 120 may be two separate elements which may be coupled in a fixed manner or alternatively may be manufactured as a single unit. Fluoroscopic imaging device 124 may include a single imaging device or more than one imaging device.

As noted above, workstation 11 may be any suitable computing device including a processor and storage medium, wherein the processor is capable of executing instructions stored on the storage medium. Workstation 11 may further include a database configured to store patient data, image data sets, white light image data sets, computed tomography (CT) image data sets, magnetic resonance imaging (MRI) image data sets, fluoroscopic data sets including fluoroscopic images and video, fluoroscopic 3D reconstruction, navigation plans, and any other such data. Although not explicitly illustrated, workstation may include inputs, or may otherwise be configured to receive, CT data sets, fluoroscopic images/video and other data described herein. Additionally, workstation 11 may be connected to one or more networks through which one or more databases may be accessed.

The bronchoscope 102 may include one or more pull-wires which can be used to manipulate the distal portion of the catheter. Pull-wire systems are known and used in a variety of settings including manual, power assisted, and robotic surgeries. In most pull-wire systems at least one but up to six and even ten pull wires are incorporated into the bronchoscope 102 and extend from proximate the distal end to a drive mechanism located at a proximal end. By tensioning and relaxing the pull-wires the shape of the distal portion of the catheter can be manipulated. For example, in a simple two pull-wire system by relaxing one pull-wire and retracting an opposing pull-wire the catheter may be deflected in the direction of the retracting pull-wire. Though certain pull-wire systems are described here in detail, the present disclosure is not so limited, and the manipulation of the bronchoscope 102 may be achieved by a variety of means including concentric tube systems and others that enable movement of the distal end of the bronchoscope 102. Further though a motor assisted/robotic system is described in detail, the same principals of extension and retraction of pull wires may be employed by manual manipulation means to change the shape of the distal portion of the catheter without departing from the scope of the present disclosure.

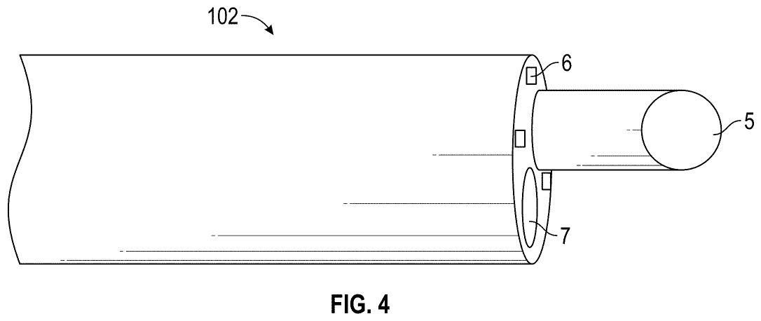

FIG. 4 depicts an alternative bronchoscope 102. The bronchoscope 102 includes an imager 5 which extends beyond the distal end of the bronchoscope 102. The imager is mounted on a swivel which allows for movement in either or both the up/down directions or the left/right directions, and may be configured to capture images both in the forward directions and in the backwards directions. For example, if the imager 5 can swivel in the up/down direction 135 degrees relative to the forward direction, a scan of 270 degrees is achieved and images in the backwards direction of the endoluminal network can be captured.

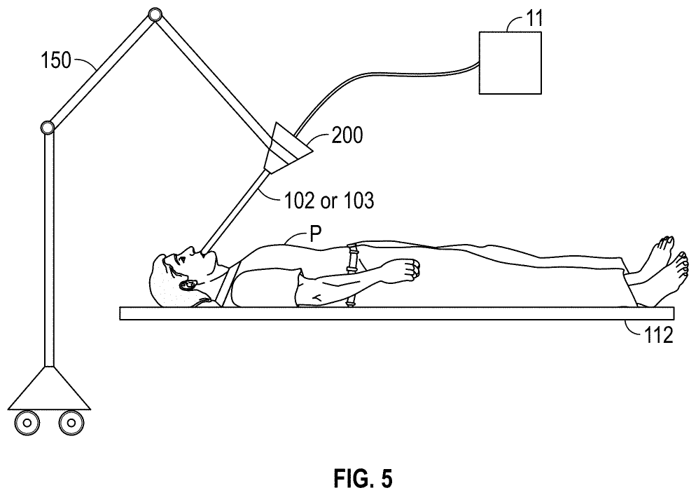

FIG. 5 depicts an exemplary motor assisted or robotic arm 150 including a drive mechanism 200 for manipulation and insertion of the bronchoscope 102 or a catheter 103 (described in greater detail below) into the patient. The workstation may provide signals to the drive mechanism 200 to advance and articulate the bronchoscope 102 or catheter 103. In accordance with the present disclosure the workstation 11 receives the images and compiles or manipulates the images as disclosed elsewhere herein such that the images, compiled images, 2D or 3D models derived from the images can be displayed on a display 18.

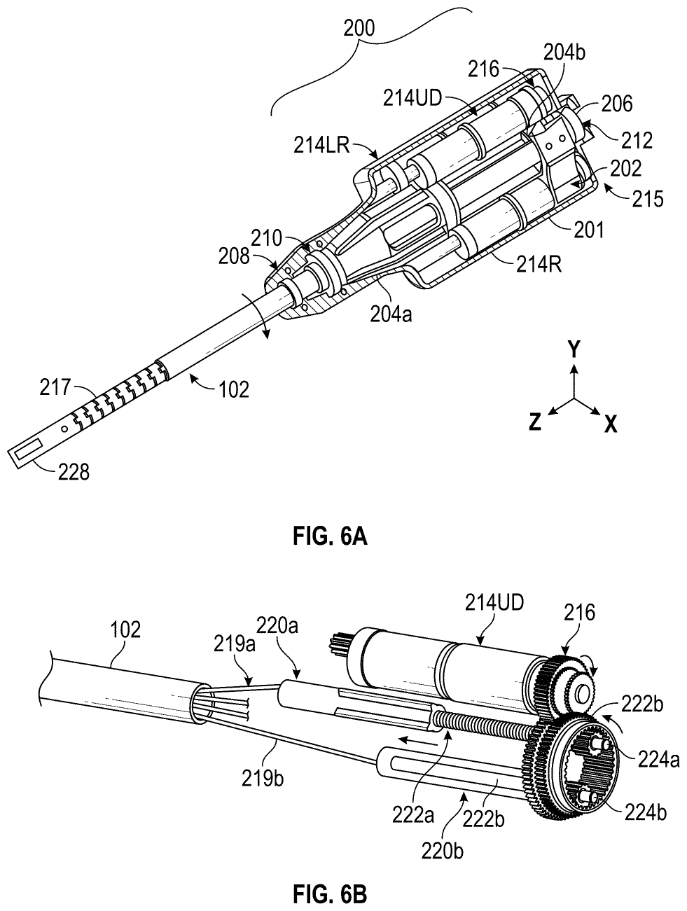

In accordance with the present disclosure, the drive mechanism receives signals generated by the workstation 11 to drive the bronchoscope 102 (e.g., extend and retract pull-wires) to ensure navigation of the airways of the lungs and to acquire images from the desired airways and in some instances all the airways of the patient into which the bronchoscope 102 will pass. One example of such a device can be seen in FIG. 6A which depicts a housing including three drive motors to manipulate a catheter extending therefrom in 5 degrees of freedom (e.g., left right, up, down, and rotation). Other types of drive mechanisms including fewer or more degrees of freedom and other manipulation techniques may be employed without departing from the scope of the present disclosure.

FIG. 6A depicts the drive mechanism 200 housed in a body 201 and mounted on a bracket 202 which integrally connects to the body 201. The bronchoscope 102 connects to and in one embodiment forms an integrated unit with internal casings 204a and 204b and connects to a spur gear 206. This integrated unit is, in one embodiment rotatable in relation to the housing 201, such that the bronchoscope 102, internal casings 204a-b, and spur gear 206 can rotate about shaft axis “z”. The bronchoscope 102 and integrated internal casings 204a-b are supported radially by bearings 208, 210, and 212. Though drive mechanism 200 is described in detail here, other drive mechanisms may be employed to enable a robot or a clinician to drive the bronchoscope 102 to a desired location without departing from the scope of the present disclosure.

An electric motor 214R, may include an encoder for converting mechanical motion into electrical signals and providing feedback to the workstation 11. Further, the electric motor 214R (R indicates this motor if for inducing rotation of the bronchoscope 102) may include an optional gear box for increasing or reducing the rotational speed of an attached spur gear 215 mounted on a shaft driven by the electric motor 214R. Electric motors 214LR (LR referring to left-right movement of an articulating portion 217 of the bronchoscope 102) and 214UD (referring to up-down movement of the articulating portion 217), each motor optionally includes an encoder and a gearbox. Respective spur gears 216 and 218 drive up-down and left-right steering cables, as will be described in greater detail below. All three electric motors 214 R, LR, and UD are securely attached to the stationary frame 202, to prevent their rotation and enable the spur gears 215, 216, and 218 to be driven by the electric motors.

FIG. 6B depicts details of the mechanism causing articulating portion 217 of bronchoscope 102 to articulate. Specifically, the following depicts the manner in which the up-down articulation is contemplated in one aspect of the present disclosure. Such a system alone, coupled with the electric motor 214UD for driving the spur gear 216 would accomplish articulation as described above in a two-wire system. However, where a four-wire system is contemplated, a second system identical to that described immediately hereafter, can be employed to drive the left-right cables. Accordingly, for ease of understanding just one of the systems is described herein, with the understanding that one of skill in the art would readily understand how to employ a second such system in a four-wire system. Those of skill in the art will recognize that other mechanisms can be employed to enable the articulation of a distal portion of a bronchoscope 102 and other articulating catheters may be employed without departing from the scope of the present disclosure.

To accomplish up-down articulation of the articulating portion 217 of the bronchoscope 102, steering cables 219a-b may be employed. The distal ends of the steering cables 219a-b are attached to, or at, or near the distal end of the bronchoscope 102. The proximal ends of the steering cables 219a-b are attached to the distal tips of the posts 220a, and 220b. The posts 220a and 220b reciprocate longitudinally, and in opposing directions. Movement of the posts 220a causes one steering cable 219a to lengthen and at the same time, opposing longitudinal movement of post 220b causes cable 219b to effectively shorten. The combined effect of the change in effective length of the steering cables 219a-b is to cause joints a forming the articulating portion 217 of bronchoscope 102 shaft to be compressed on the side in which the cable 219b is shortened, and to elongate on the side in which steering cable 219 a is lengthened.

The opposing posts 220a and 1220b have internal left-handed and right-handed threads, respectively, at least at their proximal ends. Housed within casing 1204b are two threaded shafts 222a and 222b, one is left-hand threaded and one right-hand threaded, to correspond and mate with posts 220a and 220b. The shafts 222a and 222b have distal ends which thread into the interior of posts 220a and 220a and proximal ends with spur gears 224a and 224b. The shafts 222a and 222b have freedom to rotate about their axes. The spur gears 224a and 224b engage the internal teeth of planetary gear 226. The planetary gear 226 also includes external teeth which engage the teeth of spur gear 218 on the proximal end of electric motor 214UD.

To articulate the bronchoscope in the upwards direction, a clinician may activate via an activation switch (not shown) for the electric motor 214UD causing it to rotate the spur gear 218, which in turn drives the planetary gear 226. The planetary gear 226 is connected through the internal gears 224a and 224b to the shafts 222a and 222b. The planetary gear 226 will cause the gears 224a and 224b to rotate in the same direction. The shafts 222a and 222b are threaded, and their rotation is transferred by mating threads formed on the inside of posts 220a and 220b into linear motion of the posts 220a and 220b. However, because the internal threads of post 220a are opposite that of post 220b, one post will travel distally and one will travel proximally (i.e., in opposite directions) upon rotation of the planetary gear 226. Thus, the upper cable 219a is pulled proximally to lift the bronchoscope 102, while the lower cable 219b must be relaxed. As stated above, this same system can be used to control left-right movement of the end effector, using the electric motor 214LR, its spur gear 216, a second planetary gear (not shown), and a second set of threaded shafts 222 and posts 220 and two more steering cables 219. Moreover, by acting in unison, a system employing four steering cables can approximate the movements of the human wrist by having the three electric motors 214 and their associated gearing and steering cables 219 computer controlled by the workstation 11.

Though generally described above with respect to receiving manual inputs from a clinician as might be the case where the drive mechanism is part of a motorized hand-held bronchoscope system, the present disclosure is not so limited. In a further embodiment, the drive mechanism 200 is part of a robotic system including robotic arm 150 (FIG. 5) for navigating the bronchoscope 102 or a catheter 103 to a desired location within the body. In accordance with this disclosure, in instances where the drive mechanism is part of a robotic bronchoscope drive system, the position and orientation of the distal portion of the bronchoscope 102 or catheter 103 may be robotically controlled.

The drive mechanism may receive inputs from workstation 11 or another mechanism through which the surgeon specifies the desired action of the bronchoscope 102. Where the clinician controls the movement of the bronchoscope 102, this control may be enabled by a directional button, a joystick such as a thumb operated joystick, a toggle, a pressure sensor, a switch, a trackball, a dial, an optical sensor, and any combination thereof. The computing device responds to the user commands by sending control signals to the motors 214. The encoders of the motors 214 provide feedback to the workstation 11 about the current status of the motors 214.

In a further aspect of the present disclosure the bronchoscope 102 may include or be configured to receive an ultrasound imager 228. The ultrasound imager 228 may be a radial ultrasound transducer, a linear ultrasound transducer, a capacitive micromachined ultrasonic transducer, a piezoelectric micromachined ultrasonic transducers, or others without departing from the scope of the present disclosure. In accordance with the present disclosure, following the navigation of the bronchoscope 102 to a location an ultrasound imaging application may be engaged.

Employing the systems described herein the bronchoscope 102 or the capsule 40 may be navigated through the endoluminal network (e.g., the airways) of the patient. The imagers 46 or imager 5 and light pipes 2 and reflectors 3 are configured to capture images of the endoluminal network from two perspectives. One such perspective is a forward perspective (e.g., the perspective from the endoscope 1 and in the direction of travel when proceeding from the trachea towards the alveoli (e.g., from proximal to distal). The second perspective is one that is opposite the direction of travel of the endoscope, that is a backwards view or backwards perspective. Capturing both of these image data sets (i.e., the forward image data stream and the backwards image data stream) ensures that any pathology or areas of interest which might be located at a location not immediately viewable with the bronchoscope 102 when considering just the forward perspective.

While navigating the endoluminal network images are captured. These images may be stored in storage unit 19 or the image database 10. One or more applications stored in a memory on workstation 11 can be employed to analyze the images. These applications may employ one or more neural networks, artificial intelligence AI, or predictive algorithms to identify those images which display indicators of some pathology or other items of interest. Further, the applications may be employed to identify features and landmarks of the endoluminal network.

According to an embodiment of the present disclosure, the data processor 14 may include an editing filter 22 for editing a moving image stream. Editing filter 22 may be an editing filter processor and may be implemented by data processor 14. While the editing filter is shown in FIG. 1 as being separate from and connected to processor 14, in some embodiments editing filter may be a set of code or instructions executed by, for example, processor 14. Editing filter 22 may be or include one or more dedicated processors. The editing filter 22 may generate a subset of the original input set of images (the remaining images may be removed or hidden from view). The editing filter 22 may evaluate the degree or occurrence in each frame of each of a plurality of pre-defined criteria from logical database 20. The editing filter 22 may select only a subset of images according to the predefined criteria, constraints, and rules provided by the logical database 20, to form a subset of images of interest. Preferably, the editing filter 22 may select for display only a portion of some images, for example a portion of an image which matches a predefined criteria, e.g. the portion of the image which received a high score according to the one or more rules or criteria provided in logical database 20. In selecting a portion, the portion may be made to fit a frame, and thus the portion may include non-selected image data.

Further, editing filter 22 may select images or portions of images from one or more image streams captured by one or more of the imager 5 and light pipes 2 and reflectors 3 (or imagers 46). The image streams may be processed separately, for example, each stream may be processed as a separate stream and images may be independently selected from each stream captured by a single imager 46. In other embodiments, streams may be merged, for example images from two or more streams may be sorted chronologically according to the capture time of the images and merged into a single stream. Other sorting methods are possible, for example based on different image parameters such as similarity between images or based on the score assigned to the image portions by the pathology or abnormality detectors. The merged stream may be processed as one stream (e.g., editing filter 22 may select images from the merged stream instead of separately from each stream).

There are many factors to consider for efficiently reviewing in vivo images, various of which may affect the editing used in different embodiments. In one embodiment, the set of displayed images includes as many images as possible, which may be relevant to generate a correct diagnosis of the patient's condition by a health professional. It may be less desirable to omit certain highly informative images from the set of displayed images, to ensure correct diagnosis. Pathologies or abnormalities in human tissue have a very wide range of manifestation, making them in some cases difficult to detect. Accordingly, the editing filter 22 may select frames or portions of frames based on a specific predetermined criterion, or on a combination of a plurality of pre-determined criteria.

The pre-determined criteria may include, for example, a measure or score of one or more pathology detections and/or anatomical landmark detections (e.g., lesion detector, blood detector, ulcer detector, anomaly detector, bifurcation detector, etc., which are determined based on color, texture, structure or pattern recognition analysis of pixels in the frames), a measure or score of visibility or field of view in the frame of biological tissue which may be distorted or obscured by features such as shadows or residue, the estimated location or region of the capsule (e.g., a higher priority may be assigned to frames estimated to have been captured in a particular region of interest), frame capture or transmission rate, or any combination or derivation thereof. In some embodiments, the criteria used may be converted to scores, numbers or ratings before being evaluated with other criteria, so that the various criteria may be compared against each other.

The editing filter 22 may compute and assign one or more measures, ratings or scores or numbers to each frame based on one or more pre-determined criteria. In some embodiments, a single criterion may be used to select a subset of images for display containing only image portions pertaining to the selected criterion. For example, each image may be scanned for lesions by a lesion detector. The lesion detector may produce a score of the probability of a lesion existing in the image, and may also provide estimated boundaries of that lesion in the image. Based on the estimated boundaries, only the relevant portion of the image may be extracted into the subset of selected images for display.

In some embodiments, several different subsets of image portions may be selected for display, each subset pertaining to a different criterion. For example, one subset of images may include all images or portions of images associated with a high score or probability of lesion existence, while another subset of images may present all image or portions thereof relevant to or associated with blood or redness detection in the images. In some embodiments, the same image may be a part of two or more subsets of different criteria. It may be beneficial for a health care professional to view a subset of images including all image portions pertaining to the same symptom or pathology, since such view may increase the chance of correct diagnosis, e.g. quickly finding the true positives (e.g. the actual lesions) suggested by the filter 22, and easily identifying the false positives (portions of images which were wrongly detected by the filter 22 as lesions). Such a view may increase the positive predictive value (or precision rate, which is the proportion of patients with positive test results who are correctly diagnosed) of the medical procedure. While the results of the filter 22 do not change, the specific method of display may cause the physician or health care professional to see the pathologies more easily on one hand, and to quickly pass over images which are clearly not pathologies (the false positives) on the other hand, thus improving the detection of true positives, and reducing the overall diagnosis time invested in a single case.

A score, rating, or measure may be a simplified representation (e.g., a derived value or rating, such as an integer 0-100) of more complex characteristics of an image or a portion of an image (e.g., criteria, such as, color variation, appearance of certain textural or structural patterns, light intensity of the image or portions thereof, blood detection, etc.). A score may include any rating, rank, hierarchy, scale or relative values of features or criteria. Typically, a score is a numerical value, for example, a number from 1 to 10, but need not be limited as such. For example, scores may include, for example, letter (A, B, C, . . . ), signs or symbols (+, −), computer bit values (0, 1), the results of one or more decisions or conditions (yes no), for example, indicated by the status of one or more computing flags. Scores may be discrete (non-continuous) values, for example, integers, a, b, c, etc., or may be continuous, for example, having any real value between 0 and 1 (subject to the precision of computer representation of numbers). Any interval between consecutive scores may be set (e.g., 0.1, 0.2, . . . , or 1, 2, . . . , etc.) and scores may or may not be normalized.

Scores for each frame or portion thereof may be stored with the frames in the same database (e.g., image database 10). The scores may be defined, e.g., in a header or summary frame information package, with the data in an initial image stream or with frames copied to a second edited image stream. Alternatively or additionally, the scores may be stored in a database separate from the images (e.g., logical database 20) with pointers pointing to the images. The scores in separate database may be stored with associated predefined criteria, constraints, and rules to form a subset of selected image portions.

By using a score, the quantity of data used to represent the complex characteristics of the image may be reduced and therefore the complexity and computational effort of image comparisons is likewise reduced. For example, the editing filter 22 may attempt to determine if a criterion or feature is more visible in a portion of image A than in a portion of image B and then if the criterion or feature is more visible in a portion of image B than in a portion of image C. Without scores, the content of image B may be evaluated twice, once for comparison with image A and then again for comparison with image C. In contrast, using scores, according to embodiments of the invention, the content of each image need only be evaluated once with respect to each criterion to determine the score of the image. Once a score is assigned to image B or a portion thereof, a simple numerical comparison of scores (e.g., greater than, less than or equal to) may be executed to compare the image frame with both images A and C. Using a score to compare and select images may greatly reduce at least the number of times the content of an image is evaluated and thus the computational effort of image comparisons.

In one embodiment, the editing filter 22 may assign a single combined score, e.g., a scalar value, rating each frame or group of frames based on combined frame properties associated with two or more of the plurality of pre-determined criteria. The scores may be, for example, a normal or weighted average of frame values for each of the two or more pre-determined criteria. In one example, each frame may have a score, s1,s2,s3, . . . , assigned for each pre-determined criteria, 1, 2, 3, . . . , and the combined frame score, S, may be an average of scores, S=(s1+s2+s3)/c, where c is a scaling factor, or a weighted average, S=(w1*s1+w2*s2+w3*s3)/c, where w1, w2, and w3, are respective weights for each pre-defined criteria. In another example, the combined frame score, S, may be a product of scores, S=(s1*s2*s3)/c or S=(s1*s2+s2*s3+s1*s3)/c.

In another embodiment, the editing filter 22 may store each score individually for each individual criterion. For example, each frame may have a “score vector,” S=(s1,s2,s3, . . . ), where each coordinate of the score vector provides a value for a different pre-defined criteria for the frame so that each criteria may be separately used, evaluated, and analyzed. By separating scores for each criterion, the editing filter may quickly compare scores for different combinations of criteria, for example, using vector operations. For example, when a subset of criteria (e.g., criteria 2 and 5) are selected to produce the subset of images for display, the editing filter 22 may quickly retrieve the corresponding scores (e.g., the second and fifth coordinates of the score vector S=(s2,s5)). A score vector may refer to any representation or storage that separates individual scores for each criterion, for example, such as a table or data array. In a score vector, the scores may be all in the same units (e.g., a number), but need not be.

The editing filter 22 may assign frames weighted scores, in which larger weights may be assigned for some pre-defined criteria than others. For example, since a large lesion (e.g., at least 6 mm in diameter) is more significant for diagnosis than a small lesion (e.g., 1 mm in diameter), the weight assigned to the large lesion score may be greater than the weight assigned to the small lesion score. While in some embodiments lesions are discussed, other pathologies, and other features, may be detected, rated, or scored. The score for each criterion may be weighted or combined in any suitable manner. In one embodiment, the weight of one score may affect the weight(s) of one or more other scores. For example, when one score exceeds a predetermined threshold, the weights of other scores may be changed in the combined score or the score may be added (e.g., the weight being changed from zero to one or more) or removed (e.g., the weight being changed from one to zero) from the combined score. In another embodiment, different weights for one or more scores may be used for different respective regions of the endoluminal network. For example, when a capsule is in (or is estimated to be) the trachea (e.g., indicated by the location score or probability of being in the trachea), a score indicating the tissue visibility may be given less weight because the relatively wide passage of the trachea rarely obscures tissue visibility, thereby making the score less of a defining feature than other scores.

The scores or measures may be absolute or relative to each other. The absolute score(s) for each frame or portion of frame may be a value associated with the criteria for the single frame. The relative score(s) for each frame or for a portion of frame may be a change in the value associated with the criteria relative to the value associated with the criteria for a previous or adjacent frame. Both absolute and relative scores may or may not be scaled (normalized). Scores may be scaled with a different scaling factor, for example, for images captured or estimated to be captured within each region of the endoluminal network, each segment of the image stream or for each different frame capture and/or transmission rate.

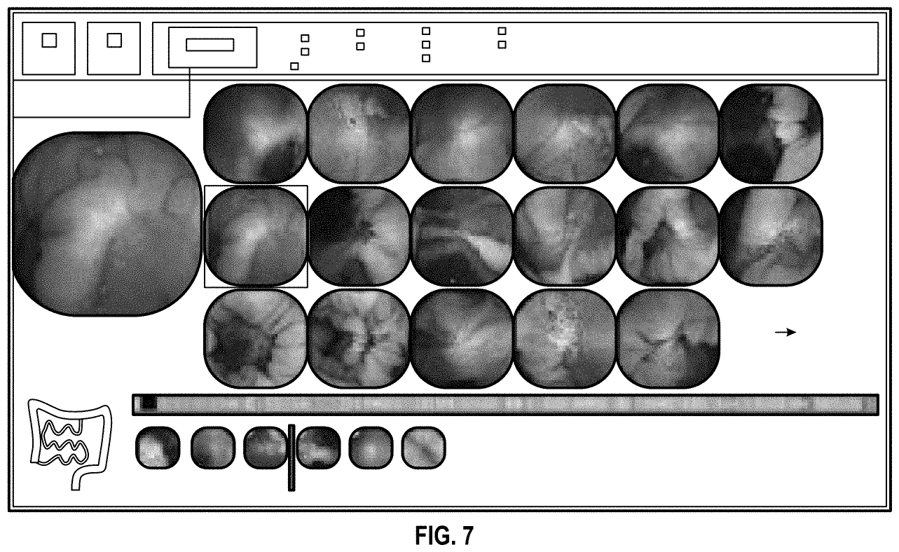

The particular pre-determined criteria and their measures, ratings or scores used for selecting a subset of images for display in a two-dimensional tiled array layout may be preset (e.g., by a programmer or at a factory), automatically selected by the data processor 14 or the editing filter 22 itself and/or manually selected by a user (e.g., using input device 24). In one embodiment, the editing filter 22 may always use one or more default criteria, for example, unless modified by a user. An editing graphical user interface (GUI) (FIG. 7) may enable a user to select from a plurality of possible criteria, from which a user may choose one or more. In another embodiment, the pre-determined criteria may be semi-automatically selected by a processor and/or semi-manually selected by a user. For example, the user may indirectly select pre-determined criteria by selecting the desired properties or constraints associated with the movie, such as a maximum movie length (e.g., 45 minutes or 9000 images), a review mode (e.g., preview movie, quick view mode, pathology detection mode, colon analysis mode, small bowel analysis mode, etc.), or other editing constraints. These parameters may in turn trigger the automatic selection of pre-determined criteria by a processor that meet the user-selected constraints.

The editing filter 22 may determine whether a frame or a portion of a frame corresponds to the selection criteria, and assign a score based on the level of correspondence. The editing filter 22 may compare the scores of each image portion to a predetermined threshold value or range. The editing filter may select for display each frame with a score exceeding (or lower than) the predetermined value or within the predetermined range for display. Accordingly, the editing filter 22 may not select for display (or may select for deletion) each frame with a score below the predetermined value or outside the predetermined range. In some embodiments, the score threshold may not be predetermined, but instead may be automatically calculated by editing filter 22 and/or data processor 14. The scores may be calculated, for example, based on the number of images in the original image stream (so that a predetermined number of input images satisfy the threshold or a predetermined percentage of input images satisfy the threshold), based on the number of images required in the selected set of images (so that a predetermined number of selected images satisfy the threshold), or based on a time limit for display of the selected set of images (so that the number of images that satisfy the threshold form a selected set of images with a viewing time of less than or equal to a predetermined time, for example when viewing the selected set of images in a standard or average display rate). In some embodiments a user may set these parameters, while in other embodiments the parameters may be predetermined or automatically generated by editing filter 22.

In some embodiments, the editing filter 22 may crop an image, to leave the relevant portion of the image (possibly within a frame such as a square or rectangle), and store it as a selected portion for display in the spatial layout. The original image or frame may be cropped based on the detected borders or edges of the pathology detector that caused the frame to be selected. For example, the original frame may be selected after receiving, for example, a high score by the lesion detector. The lesion detector may detect a lesion in a frame and determine or estimate the lesion's edges. The editing filter may crop the original image and leave only the lesion (and some surrounding pixels) in the selected image portion, including the lesion's edges as determined by the detector. Similarly, frames which receive high scores based on other pathology detectors, may be cropped according to the determined edges or estimated borders of the detected pathology. In some cases, more than one pathology may be detected in a single frame, and multiple portions of the same frame may be selected for display in the spatial layout.

In some embodiments, the editing filter 22 may select images pertaining to certain anatomical landmark points in the body lumen traversed by the capsule 40, such as the entrance to one or more named bifurcations of the lungs. Other anatomical landmarks may be detected and selected for display by editing filter 22.

The editing filter 22 may include or may be embodied in one or more execution units for computing and comparing scores, such as, for example, an arithmetic logic unit (ALU) adapted executing arithmetic operation, such as add, multiple, divide, etc. The editing filter 22 may be or may be embodied in a processor (e.g., hardware) operating software. The editing filter 22 may include one or more logic gates and other hardware components to edit the original image stream to generate the edited image stream. Alternatively or additionally, the editing filter 22 may be implemented as a software file stored for example in logic database 20 or another memory, in which case a sequence of instructions being executed by for example data processor 14 results in the functionality described herein.

The original image stream may be divided into segments. A segment may be defined based on different parameters, such as a time parameter (e.g. a segment captured during one minute), a number of frames (e.g., 1000 consecutive frames), or frames associated with a detected or estimated anatomical region or landmark point in the body lumen. In some embodiments, more than one parameter may be used concurrently to define a segment. For example, a trachea segment of the original image stream may be represented by a number of images larger than a predetermined threshold in the subset of images. Each segment may be represented by at least a predetermined number of images or image portions (for example, one or two) selected for display in the spatial layout. The selected subset of images may be displayed in a rectangular tiled array layout on the screen or display 18, as shown in FIG. 7.

A layout unit 28 may determine the arrangement of the image portions selected by editing filter 22 on the screen or display 18. While the layout unit 28 is shown in FIG. 1 as being separate from and connected to processor 14, in some embodiments layout unit 28 may be a set of code or instructions or an application executed by processor 14. Layout unit 28 may be or include one or more dedicated processors. Layout unit 28 may select or generate a spatial arrangement of a subset of the original image stream, including selected images or portions thereof. The spatial arrangement of the subset of image portions on the display 18 may be predetermined or may be selected by a user.

A user may prefer to view a layout which includes only the relevant portions of the selected frames, which comply with the predetermined or selected criteria or rules, for example portions of frames which receive a score which is higher or lower than a certain threshold determined for each type of selection criterion. For example, a rectangular tiled array made of 100 images may be generated for display, e.g. 10 rows and 10 columns of relevant portions of selected frames from the original input image stream. Preferably, all portions are arranged adjacent to each other, creating a tiled array with no white spaces or background spaces between the portions of frames. Such an arrangement may increase the visibility of pathological tissue if it exists in the displayed layout, since the tiled array may produce a homogenous view of the suspected image portions, and pathology may be prominent or may stand out in such distribution or arrangement. The selected image portions may be resized, for example by the layout unit 28, to an appropriate dimension or size, based on the selected layout, spatial arrangement and/or grid. In some embodiments the selected image portions may be resized to a single uniform dimension, while other embodiments allow for resizing or scaling the image portions displayed in the layout into different dimensions.

Relevant portions of the selected frames, as detected by the editing filter 22, may be arranged by layout unit 28 to maximize evenness or uniformity of the displayed array. The layout unit 28 may apply a filter (e.g., a “homogenizing” filter) to remove portions of frames which create an uneven, heterogeneous or noisy frame layout, or portions which have a disturbing effect on the eye of a user. For example, the layout unit 28 may minimize the occurrence of portions of images which may unnecessarily attract the physician's attention, such as dark portions of frames or portions with bad visibility due to intestinal juices or content, turbid media, bile, bubbles, image blurring, or other causes. Image portions which have been detected by editing filter 22 as complying with the selected criteria, may be subject to further processing or cropping, based on the detection of areas with bad visibility within the selected image portion. Portions of frames with bad visibility may be cropped from the displayed image portion, or the image portion may be removed completely from the displayed layout. Consequently, the occurrence of insignificant or irrelevant portions of images may be minimized in the displayed array of image portions, and the positive prediction and diagnosis value of the capsule procedure may increase.

The layout unit 28 may include or be embodied in one or more execution units for computing and comparing scores, such as, for example, an arithmetic logic unit (ALU) adapted executing arithmetic operation, such as add, multiple, divide, etc. The layout unit 28 may be a processor (e.g., hardware) operating software. The layout unit 28 may include one or more logic gates and other hardware components to edit the original image stream to generate the edited image stream. The layout unit 28 may be implemented as a software file stored for example in logic database 20 or another memory, in which case a sequence of instructions executed by for example data processor 14 result in the functionality described herein.

Once editing filter 22 selects the image portions, they may be merged by layout unit 28 to form a tiled array layout or grid. The resolution or number of image portions displayed in the layout may be predetermined or may be selected by a user according to his/her preference.

Layout unit 28 may receive a set of selected image portions and may determine which of the selected image portions will be displayed in each layout page. For example, the number of selected image portions from the original image stream may be 5,000. The generated or selected spatial arrangement of the layout pages may include 100 image portions in each layout page. Thus, 50 non-overlapping layout pages, each comprising different selected image portions, may be generated by the layout unit 28 and displayed to the user, for example sequentially (chronologically) or using a different sorting method such as a degree of similarity score between the selected portions. Typically, the physician may prefer keeping chronological order between the different layout pages, while the internal arrangement of the portions in a layout page may not be necessarily chronological. In another embodiment, the segmentation of image portions to specific layout pages may be determined based on the degree of similarity between images or based on scores of different criteria which may be generated by the editing filter 22.

Thus, by acquiring the images a physician and workstation 11 is provided with image data that can be used for navigation of a catheter 103 or other tool to an area of interest in the endoluminal network identified in the image data. For example, a manual, motorized, or robotic catheter 103 may be navigated in the endoluminal network in a similar manner as the bronchoscope 102. Indeed, in at least one embodiment the catheter 103 is substantially the same as bronchoscope 102, with perhaps different imagers 5 and a larger working channel to accommodate biopsy or therapeutic tools. Where the catheter 103 also includes an imager 5 (as described in connection with endoscope 1, above) the images acquired by the imager of the catheter 103 may be compared to those captured by the capsule 40 or bronchoscope 102. The comparison of the images reveals proximity of the catheter 103 to the pathologies, lesions and landmarks within the lungs.

In one embodiment, an artificial intelligence associated the workstation 11 can analyze the original images acquired from capsule 40 or bronchoscope 102 and based on landmarks determine a pathway to an area of interest (e.g., a pathology or lesion). This pathway can then be utilized to enable efficient navigation to the pathologies and lesions identified in those images. As a result upon navigation of the diagnostic or therapeutic catheter 103 the display 18 can provide a GUI that alerts the clinician as to which airway to navigate the catheter 103 in as landmarks are identified in the real time images captured by the imager 5 of the catheter 103 and compared to those images previously captured, for example by bronchoscope 102. The GUI may also provide distance and direction information to lesions or pathology. Still further, the pathway can be employed by the workstation 11 to drive the robotic arm 150 and the drive mechanism 200 to navigate the catheter 103 along the pathway with the clinician merely observing the progress of the catheter 103 to the areas of interest.

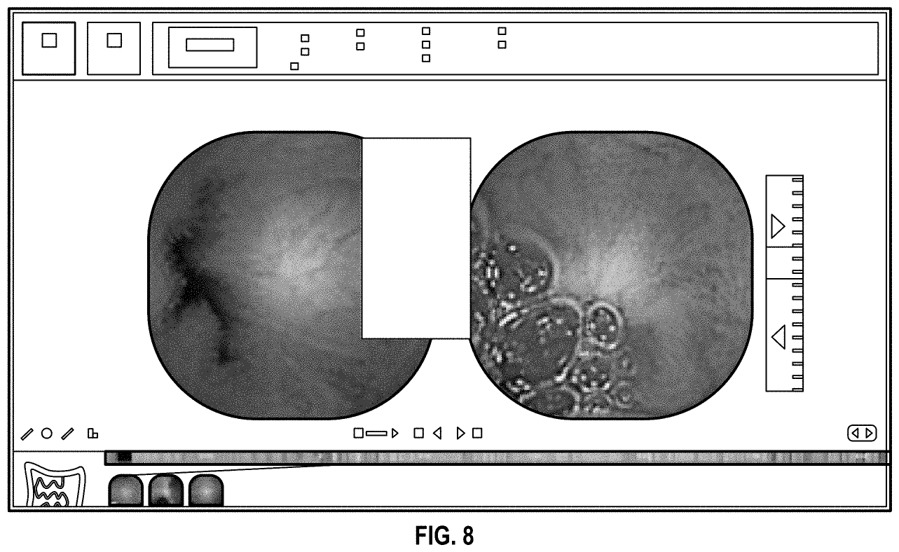

In some embodiments the real time images acquired by imager 5 of the catheter 103 can be displayed simultaneously with the prior images acquired by the bronchoscope 102 (e.g., providing a side-by-side comparison) as depicted in FIG. 8. Such comparison is useful prior to diagnostic or therapy to confirm navigation of the catheter 103 to the same location as identified in the images captured by bronchoscope 102. Further, this side-by-side comparison allows for monitoring of the change in condition of an area of interest over time, and in instances where a therapy has been undertaken to allow for the analysis of the healing response experienced at a specific location. Still further, relying on the accurate detection of location of the catheter 103 based on the comparison of the forward images, the backward images captured by the bronchoscope 102 (e.g., from light pipes 2 and reflectors 3) can be displayed as well. This allows for further information regarding a particular location within the endoluminal network to be assessed even when catheter 103 does not include such backward facing imaging capabilities. This may be particularly useful to ensure that the margins of the lesion are in fact in view in the real time images.

Still further, the real time images captured by the imager 5 of the catheter 103 may be assessed by the workstation 11 in a similar manner as described above with respect to bronchoscope 102 and capsule 40 to identify any new lesions or changes to lesions that might have manifest themselves since the navigation of the bronchoscope 102 or the capsule 40. The imaging capabilities of the imager 5 of the catheter 103 may be different than the imaging capabilities of the bronchoscope 102. This multispectral imaging can take a variety of forms including white light, infrared, near infrared, tunable laser light and others without departing from the scope of the present disclosure. For example, the imager 5 may be a near infrared (NIR) imager which can be employed to detect autofluorescence and other aspects of the tissue being imaged. The data collected by this second imaging capability may be added to the image data from the bronchoscope 102 to create a composite image data set. Again, neural networks or AI may be employed in analyzing these NIR image data sets and provide indicia on the GUI presented on the display 18. If tunable laser imaging is employed double imaging spectrography (e.g., double blue) techniques may also be employed and analyzed by the AI in accordance with the present disclosure. As will be appreciated each image data set, regardless of the spectrum in which it is acquired may be analyzed by an AI or neural network to identify pathologies and lesions and bring these to the clinician's attention. This may be done in real time as the bronchoscope 102, catheter 103, or capsule 40 is navigating the airways, or may be a process which runs separate from the procedures but is associated with one or more applications stored in a memory on workstation 11 or on a separate worksation.

As the images are initially acquired, the position of the bronchoscope 102 or capsule 40 may be tracked by the tracking system 114 (e.g., using sensor 104). The position at which each image is acquired by the bronchoscope 102 or capsule 40 can be recorded and associated with the image. A time stamp may also be associated with image to identify the time at which the image was acquired. This data may be employed by the workstation 11 to create a two-dimensional (2D) or a three-dimensional (3D) model of the endoluminal network. The 2D model may be a series of images compiled or stitched together displayed in a flat form. In effect this would be a model which depicts the endoluminal network as if the network were cut longitudinally and laid flat. Additionally or alternatively, the 3D model generated from the images may provide a fly-through view of the endoluminal network. When presented in the GUI on the display 18, the view would be from the perspective of the imager 5 as it looks forward. The 3D model may also depict the backward view as viewed via the backward imager (e.g., the light pipes 2 and reflectors 3). The two 3D models may be simultaneously displayed on the GUI on display 18 (similar to the side-by-side display in FIG. 8) enabling viewing of aspects of the endoluminal network that might be missed by the forward viewing imager 5. Using the tracking system 114 and a sensor 4 in catheter 103, the position of the catheter 103 can be determined and thus the pathway to the areas of interest followed to allow for insertion of one or more diagnostic or therapeutic tools at the areas of interest (e.g., lesions). Regardless of whether a 2D model or 3D model or individual image frames on the GUI, any areas of interest (e.g., lesions or pathologies) identified by the AI operating in conjunction with workstation 11, or manually entered by a clinician are displayed on the GUI at the appropriate locations.

As noted above, the frame rate at which the images are captured may be variable. Employing sensor, robotics, or other means the direction of travel of the catheter 103 or the bronchoscope 102 may be captured. As the airways of the lungs are a series of lumens which form the endoluminal network, there may be a need to alter the image capture rate or the storage of images when the bronchoscope 102 is travelling in a backwards direction (e.g., in the direction of the trachea from the periphery of the lungs). In this way, when one airway of the lungs has been imaged, and the catheter 103 or bronchoscope 102 is retracted back to the nearest bifurcation, fewer images may be required or imaging may be ceased except for occasionally to confirm location and to provide guidance on when the bifurcation has been reached and to begin advancement again. Still further, imaging done by the catheter 103 may have its frame rate slowed to just what is necessary for navigation and then increased when proximate an area of interest to provide more details regarding the lesion or pathology. Reduction in frame rate reduces energy consumption of the systems and limits the amount of image data that is acquired and analyzed by the workstation 11.

Still further, when viewing either the captured images, the 2D model or the 3D model a clinician may make notes or comments. These notes and comments may be associated with a particular location in the endoluminal network. When navigating catheter 103 through the endoluminal network these notes, or comments may be presented on the GUI on display 18 when the catheter 103 is navigated to the same location at which the notes or comments were associated with.

In another aspect, when the catheter 103 is robotically driven, and the robotic system provides a further coordinate system, any position and orientation data from the original imaging (e.g., by capsule 40 or bronchoscope 102) may be updated to eliminate any imprecision in the original position and orientation data associated with a particular frame or series of frames or images.

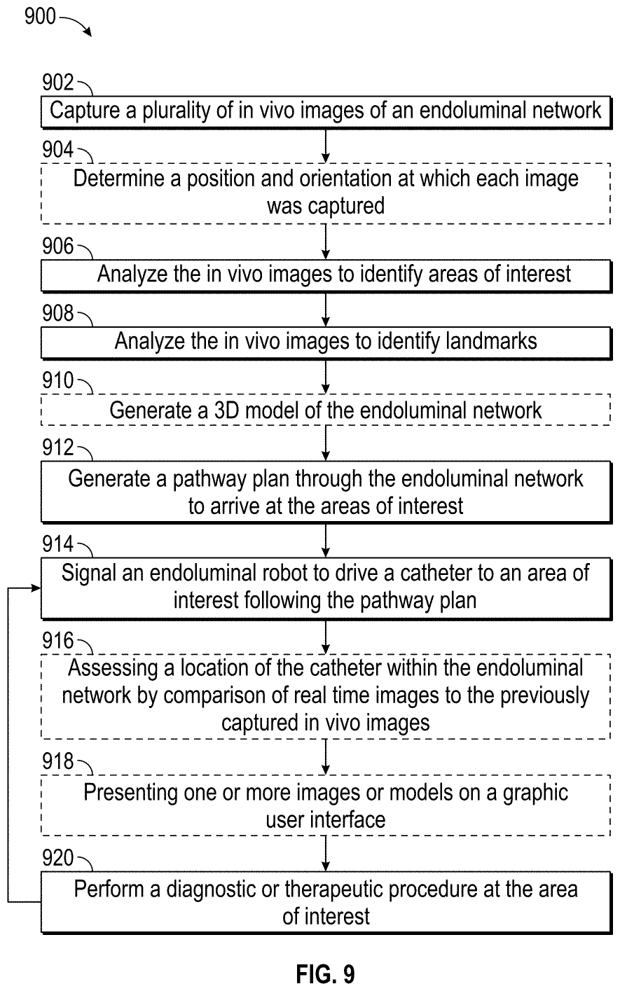

FIG. 9 details a method 900 of implementing the aspects and features described herein above. At step 902 a plurality of in vivo images is captured of an endoluminal network. These images may be captured by bronchoscope 102 or the capsule 40. At step 904 the position and orientation at which each image is captured may be determined, and as noted above associated with the image. At step 906 the in vivo images may be analyzed to identify areas of interest (e.g., pathologies, lesions, etc.). As noted above this step may be performed by an AI. At step 908 the images are analyzed to identify landmarks within the endoluminal network. Optionally at step 310 a 3D model may be generated of the endoluminal network based on one or more of the location and orientation data, the images acquired in step 902 and the landmarks identified in step 908. At step 912 a pathway is generated through the endoluminal network to arrive at the areas of interest. At step 914, an endoluminal robot is signaled and provided the data necessary to follow the pathway plan through the endoluminal network to arrive at the areas of interest. At step 916, the location of the catheter may be optionally assessed by comparison of real time images to previously captured in vivo images. At step 918, one or more of the previously captured in vivo images, the real time images, a 2D or a 3D model may be presented on a graphic user interface. At step 920, once the endoluminal robot has driven the catheter to the area of interest, a diagnostic or therapeutic procedure may be undertaken at the area of interest. If there are more areas of interest the method reverts to step 914 and iterates until all areas of interest have a diagnostic or therapeutic procedure performed on them.

Throughout this description, the term “proximal” refers to the portion of the device or component thereof that is closer to the clinician and the term “distal” refers to the portion of the device or component thereof that is farther from the clinician. Additionally, in the drawings and in the description above, terms such as front, rear, upper, lower, top, bottom, and similar directional terms are used simply for convenience of description and are not intended to limit the present disclosure. In the description hereinabove, well-known functions or constructions are not described in detail to avoid obscuring the disclosure in unnecessary detail.

While several embodiments of the present disclosure have been shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular embodiments.

What is claimed is:

1. An endoluminal navigation system comprising:
an imaging device configured for capturing images in a first direction and in a second direction substantially opposite the first in an endoluminal network; and
an image processing device configured to receive the captured images and compile the images into one or more alternative forms, the imaging processing device including a processor and memory, the memory storing thereon a software application that when executed by the processor:
reviews the captured and compiled images to identify areas of interest;
constructs a three-dimensional (3D) model from the captured images, wherein the 3D model represents a fly-through view of the endoluminal network; and
a display configured to receive compiled images or the 3D model and to present the compiled images or 3D model to provide views in both the first and the second directions, wherein the areas of interest are identified in the 3D model or images.

2. The endoluminal navigation system according to claim 1, further comprising a position and orientation sensor associated with the imaging device.

3. The endoluminal navigation system according to claim 2, wherein the position and orientation of the sensor is associated with images captured at a position and orientation and a timestamp for capture of the images.

4. The endoluminal navigation system according to claim 2, wherein the position and orientation sensor is a magnetic field detection sensor.

5. The endoluminal navigation system according to claim 2, wherein the position and orientation sensor is an inertial monitoring unit.

6. The endoluminal navigation system according to claim 2, wherein the position and orientation sensor is a flex sensor.

7. The endoluminal navigation system according to claim 1, further comprising a speed sensor determining the speed at which the imaging device is transiting the endoluminal network.

8. The endoluminal navigation system according to claim 1, wherein the imaging device is mounted on a bronchoscope.

9. A method for driving an endoluminal robot comprising:
capturing a plurality of in-vivo images of an endoluminal network;
analyzing the plurality of captured images to identify one or more areas of interest within the endoluminal network;
analyzing the plurality of captured images to identify a plurality of landmarks within the endoluminal network;
generating a pathway plan through the endoluminal network to arrive at the one or more areas of interest;
signaling an endoluminal robot to drive a catheter through the endoluminal network, following the pathway plan, to arrive at the area of interest; and
performing a diagnostic or therapeutic procedure at the area of interest.

10. The method according to claim 9, wherein the plurality of in-vivo images is captured by one or more imagers in a capsule.

11. The method according to claim 10, wherein the capsule is navigated through the endoluminal network using a magnetic field generator.

12. The method according to claim 9, further comprising stitching the plurality of captured images together to form a two-dimensional model of the endoluminal network.

13. The method according to claim 9, further comprising generating a three-dimensional (3D) model from the plurality of captured images.

14. The method according to claim 13, further comprising generating the pathway plan with reference to the 3D model.

15. A method of endoluminal imaging comprising:
inserting a bronchoscope having forward and backward imaging capabilities into an airway of a patient;
navigating the bronchoscope into the airways and capturing a plurality of images in both a forward and a backward perspective;
determining a position and orientation within the airways at which each of the plurality images was captured;
analyzing with an artificial intelligence the captured plurality of images to identity areas of interest for performance of a diagnostic or therapeutic procedure;
generating a three-dimensional (3D) model of the airways of the patient;
generating a pathway plan through the airways of the patient;
signaling an endoluminal robot to drive a catheter through the airways to the areas of interest;
assessing the position of the catheter within the airways by comparison of real-time images with previously captured forward and backward images;
presenting one or more of the real-time images or the previously captured forward and backward images or the 3D model on a graphic user interface; and
performing a diagnostic or therapeutic procedure at the area of interest.

16. The method according to claim 15, wherein the captured forward and backward images are captured by one or more imagers in a capsule.

17. The method according to claim 16, wherein the capsule is navigated through the endoluminal network using a magnetic field generator.

18. The method according to claim 15, further comprising stitching the plurality of captured forward and backwards images together to form a two-dimensional model of the endoluminal network.

19. The method according to claim 15, further comprising generating a three-dimensional (3D) model from the plurality of captured forward and backward images.

20. The method according to claim 19, further comprising generating the pathway plan with reference to the 3D model.
